(19) United States
(12) Patent Application Publication
(10) Pub. No.: US 2003/0162272 A1
(43) Pub. Date: Aug. 28, 2003

(54) Method for culturing micro-organisms in reducing conditions obtained by a gas stream

(76) Inventors: Remy Cachon, Dijon (FR); Nathalie Capelle, Dijon (FR); Charles Divies, Bondoufle Cedex (FR); Lucie Prost, Paris (FR)

Correspondence Address: Air Liquide, Suite 1800, 2700 Post Oak Boulevard, Houston, TX, 77056, US

(21) Appl. No.: 10/311,985

(22) PCT Filed: Jun. 28, 2001
(86) PCT No.: PCT/FR01/02067

(30) Foreign Application Priority Data
Jul. 4, 2000 (FR) 00 08684

(51) Int. Cl. C12P 7/14

(52) U.S. Cl. 435/162; 435/161

(57) ABSTRACT

The present invention relates to a method of culturing microorganisms which makes it possible to reduce the oxidation-reduction potential of the culture medium, characterized in that the said culture is carried out under reducing conditions obtained by a reducing gas compared with air comprising hydrogen.

It also relates to such a method for modifying the metabolic flows during the culture of the microorganisms.

It applies more particularly to the food sector and in particular to the production of alcoholic beverages.

The present invention relates to the culture of microorganisms under reducing conditions obtained by means of a gaseous stream.

It relates more particularly to the modified and/or controlled production of food products by fermentation using yeasts under reducing conditions such as alcoholic beverages, dairy products and the like.

The invention also relates to the production of biomass, in particular ferments or leavens, food yeasts, probiotic ferments and yeast extracts.

It also relates to the production of active substances useful in particular in the pharmaceutical or veterinary industry.

It relates more precisely to a process which makes it possible to reduce the oxidation-reduction potential during the culture of microorganisms, in particular during fermentation using yeasts for the preparation of the abovementioned products, making it possible to modify and/or to control the metabolic flows and therefore the composition and/or the properties of the said products.

Oxidoreductions are essential steps in the cellular anabolism and catabolism reactions for which the direction of the exchanges is determined by the oxidation-reduction potential (Eh). This is a parameter of the state of the fermentations and its variation modifies the physicochemical environment of the microorganisms whose metabolic activities and physiology it influences.

Yeasts are used very widely for the manufacture of fermented beverages.

In yeast, the redox balance of the fermentation is normally equilibrated by the production of ethanol. Furthermore, yeast uses some of the fermentable sugars to synthesize biomass and secondary products of which the principal one is glycerol, third constituent of wine after ethanol and water. The production thereof will depend on the quantity of reduced coenzymes available for the biosyntheses. During the fermentation of the sugars, the formation of ethanol and of glycerol is thus essential for maintaining the redox balance.

Other products are also synthesized in sufficiently large quantities to modify the sensory characteristics of the final product. They are essentially organic acids. The formation of the organic acids such as succinate or acetate during the fermentation also has an influence on the redox balance. The succinate is formed by the oxidative pathway via citrate and α-ketoglutarate or by the reductive pathway via oxaloacetate, such that the fermentation conditions remain essential. The acetate is also formed from acetaldehyde by an aldehyde dehydrogenase.

Yeast is also characterized by the production of volatile compounds involved in fermentative aromas, namely higher alcohols, aldehydes and esters. These products are derived from carbohydrate, lipid and nitrogen metabolisms, and because of this, their production is also dependent on a possible modification of the distribution of the flows during a variation of the redox potential.

From the metabolism of sugars, the yeast strain also synthesizes storage oligosaccharides such as glycogen and trehalose, often in response to an environmental stress. These two oligosaccharides can accumulate when the carbon is depleted in the medium but also when the strain no longer has a nitrogen or sulphur source or also during a heat or osmotic stress.

Storage sugars play an important role for improving the stability and the drying of yeasts, because of this, their concentrations constitute a critical parameter in the production of active dry yeast. Indeed, to withstand the high temperatures of the drying cycle, glycogen fractions are used to provide maintenance energy to the cell whereas trehalose for its part is used as a membrane-stabilizing factor. During the drying, the yeasts can accumulate large quantities of trehalose (10-15% of the dry weight) and moreover, the quality of dry yeasts is thought to be in direct relationship with the content of cellular trehalose. Survival to dehydration is correlated with the synthesis of trehalose and during rehydration, trehalose is degraded. In fact, trehalose binds to the phosphate groups of the membranes and by replacing the water, it stabilizes them during the dehydration. In addition to acting as storage and protective factors, these sugars can also play a role in the progression of the cell cycle at low growth rate under carbon limitation.

Taking into account the current knowledge, variations in the redox potential appear to be able to modify the distribution of the products and possibly of the metabolic intermediates.

Up until now, to modify the oxidation-reduction potential of a fermentation medium, the procedure involves either heat treatment (such as pasteurization) or addition of oxidoreductive addition molecules (such as ascorbic acid, sulphites and the like).

Nevertheless, these methods can modify the characteristics of the products obtained and cannot be systematically used in the agri-foodstuffs (oenology for example), pharmaceutical or veterinary sectors because of the standards imposed.

The objective of the invention is thus to remedy the abovementioned disadvantages and to provide a method of culturing microorganisms which makes it possible to modify the metabolic flows in the cells by varying the redox potential of the medium.

The objective of the invention is also to provide a method of fermentation using yeasts in the perspective of an agri-foodstuffs application in particular in oenology, or of a pharmaceutical or veterinary application, involving means which are not toxic and not detrimental to the final products.

Another objective of the invention is to provide such a method which makes it possible to accelerate or to simplify the methods of manufacture by fermentation.

The objective of the invention is also to provide a method which makes it possible to improve the storage life in particular of ferments or leavens.

One objective of the present invention is also to provide fermented products such as beverages having a low percentage of alcohol (wines, beers and the like) and/or modified organoleptic properties.

Another objective of the present invention is also to provide ferments, food yeasts or yeast extracts having improved properties in particular from the preservation, nutritional and/or organoleptic point of view.

To these ends, the subject of the invention is a method of culturing microorganisms which makes it possible to reduce the oxidation-reduction potential of the culture medium, characterized in that the said culture is carried out under reducing conditions obtained by a reducing gas compared with air, a reducing gas comprising hydrogen.

The subject of the invention is also a method of culturing microorganisms which makes it possible to modify the metabolic flows during the said culture, characterized in that the said culture of microorganisms is carried out under reducing conditions obtained by a reducing gas compared with air, comprising hydrogen.

Its subject is also such methods of culturing microorganisms for the production of non-aerated beverages with a reduced percentage of alcohol, in particular wine-beverages and distilled beverages, aerated alcoholic beverages, in particular semi-sparkling wines, sparkling wines, champagnes, beers and ciders, of ferments, in particular bakers' yeasts, food yeasts and yeast extracts.

Its subject is also the products obtained by the abovementioned methods.

The subject of the invention is also the use of reducing conditions obtained by a gaseous stream as mentioned above to reduce the oxidation-reduction potential of a medium for culturing microorganisms for the preparation of fermented products intended for the agri-foodstuffs industry, for the pharmaceutical or parapharmaceutical industry or for the veterinary industry.

Its subject is also the use of the abovementioned reducing conditions to modify the metabolic flows during the culture of microorganisms.

Its subject is also the use of the abovementioned reducing conditions for the preparation of the products mentioned above.

The method according to the invention will be described in greater detail below.

The inventors have demonstrated, quite surprisingly, that it was possible to vary the redox potential of a medium for culturing microorganisms by means of a reducing gaseous stream compared with air such as a gaseous stream (different from air) containing hydrogen, and that the reducing conditions thus obtained made it possible to modify the metabolic flows during the culture.

They have thus shown that it was possible to orient the metabolic flows towards the production of specific compounds and/or to modify, in a controlled manner, the properties of the products obtained.

More specifically, the inventors have shown that it was possible to modify the metabolic flows or the kinetics of the secondary fermentation during the yeast fermentation, in the context of oenological applications.

They have also shown that it was possible to increase the quantity of storage sugars produced during the culture of microorganisms for the production of biomass.

They have also demonstrated that it was possible to increase the viability of the microorganisms under reducing conditions as defined and to prolong the storage life of the ferments obtained.

According to the invention, “reducing conditions” refers to the conditions obtained with the aid of a gaseous stream which is reducing compared with air, brought into contact with a culture medium, which make it possible to reduce the oxidation-reduction potential of the said culture medium relative to the value which would exist in the absence of the said gaseous stream, that is to say in air, all things moreover being equal.

The invention thus covers the cultures in the actual reducing media (the redox potential has been reduced below 0) but also the case where the gaseous stream makes it possible to reduce the oxidation-reduction potential of an initially oxidizing medium, even if the final potential reached with the aid of the said gaseous stream remains positive per se (oxidizing medium still exists in this case in the final analysis).

It is recalled that the redox potential values depend in particular on the composition of the culture medium and its pH, the reference used to assess the reduction in the redox potential obtained in accordance with the invention having the same medium composition at a similar pH.

According to the method of the invention, the reducing conditions as defined above are obtained by means of a gas different from air and which is more reducing than the latter in the sense that it makes it possible to obtain, in the medium for culturing microorganisms, a redox potential less than that obtained under conventional culture conditions, as defined by the expression “reducing conditions”. It is composed of hydrogen alone or in the form of a mixture, it being possible for one and/or the other of these gases to be in the form of a mixture with one or more other gases called here “supplementary gas(es) acceptable from the point of view of the culture”.

The supplementary gas can thus be chosen from inert gases, in particular argon, helium, but also from oxygen, carbon dioxide and nitrous oxide and mixtures in any proportions of one or more of these gases; the supplementary gas may consist of a single gas or of a mixture of gases.

It is considered to be “acceptable from the point of view of the culture” in the sense that it does not negatively interfere with the latter and therefore allows a satisfactory or even improved development of the microorganisms.

It is in addition chosen from gases which satisfy the standards and authorizations in the field of application considered (for example oenology, food products, pharmaceutical or veterinary products and the like). This indeed extends to the standards currently known, given that they change continuously, regularly authorizing the arrival and the use of novel compounds (see for example the exemptions currently granted in France for the use of ozone).

The supplementary gas is preferably selected from carbon dioxide and oxygen as well as mixtures thereof.

When a mixture is used, the gaseous stream preferably contains at least 0.5% by volume of hydrogen, more preferably between 3 and 50% by volume of hydrogen.

For the sake of ease of use and of safety, the hydrogen content is preferably chosen less than 5%.

As will be understood on reading the preceding text, the composition of the gaseous stream may vary according to the strains used and the applications envisaged as well as according to the cost constraints which may be imposed. By way of example, there may be mentioned the preferential use of hydrogen/nitrogen mixtures for the culture of microorganisms intended for oenological applications or for the production of biomass or for the culture of lactic acid bacteria.

Typically, there may also be mentioned the use of hydrogen/oxygen mixtures for example for the production of biomass according to the nature of the microorganisms (yeasts) used.

In accordance with the invention, the method is carried out according to the culture procedures conventionally used for the microorganisms considered.

This may be in particular fermentation, using in particular yeasts, or other procedures for the growth of microorganisms according to the applications chosen.

Means for bringing the culture medium into contact with the abovementioned gaseous stream are in addition provided.

The gas according to the invention may be applied before and/or while carrying out the growth of the microorganisms, by any known means.

The method according to the invention may be carried out continuously or batchwise, the latter mode being often preferred from an industrial point of view.

As demonstrated in greater detail later in the present application, the method according to the invention makes it possible to modify the metabolic flows in the microorganisms. “Modification of the metabolic flows” is understood to mean the controlled orientation of the production during a culture of microorganisms, that is to say the obtaining of specific products possibly at the expense of other products which are normally obtained, but also the modification of the characteristics of these flows, especially from the point of view of the rate of production, the increase in pressure where appropriate, and the like.

In other words, the method makes it possible to orient and control the said metabolic flows during the culture of the microorganisms so as to obtain in the end a composition different from that which would be obtained by carrying out the same culture under conventional conditions, possibly accompanied by the production of specific novel substances which are not usually obtained during the conventional production, that is to say without contact with the reducing gas as defined according to the invention.

The method of the invention also makes it possible to modify the metabolic flows at the level of the characteristics of the reaction. Typically, there may be mentioned the case of fermentations using yeasts in a closed vessel (for example a bottle) in which the kinetics of the process of secondary fermentation can be improved under reducing conditions as described above.

The modification of the metabolic flows may also correspond to an accumulation of the storage sugars, in particular trehalose and glycogen, in the cells produced.

More specific applications of the invention are described below which should not however be considered as limiting and are given solely by way of illustration.

According to one embodiment of the invention, the method is applied to microorganisms of the type used in oenology, for the preparation of non-aerated alcoholic beverages (without secondary fermentation) such as wine-based beverages or distilled beverages.

The fermentations generally involve using yeasts of the genus Saccharomyces, which are mainly carried out in a tank (open vessel) typically batchwise.

When reducing conditions as defined according to the invention are used, beverages with a higher glycerol content are for example produced compared with the same types of beverages produced under conventional conditions, that is to say without reducing the redox potential of the medium.

The increase in the production of glycerol occurs at the expense of the production of ethanol. It is thus possible to directly obtain non-aerated low-alcohol beverages while avoiding dealcoholization by a process of extraction used up until now to produce such beverages.

The method according to the invention therefore applies in particular to the production of beverages with a reduced percentage of alcohol, for example low-alcohol wine-based beverages. In this case, it is preferable to maintain an ethanol content of at least 5% by volume in order to restore the wine aromas and flavours which are appreciated by consumers because of the retention of the volatile compounds responsible for these organoleptic properties in ethanol.

The invention may however be applied to the production of beverages with a percentage of alcohol lower than the abovementioned threshold, in which case it is possible to produce low-alcohol beverages with novel organoleptic properties.

The method according to the invention also applies to the production of beverages which, with an equivalent percentage of alcohol, have improved organoleptic properties, for example in the distilled beverage sector.

The method of the invention has the advantage of allowing a substantially complete recovery and the nondegradation of the nonvolatile compounds as well as the absence of formation of bad tastes linked to the method of treatment, in particular for conventional dealcoholization.

The method according to the invention thus makes it possible to control the characteristics of the beverages produced by fermentation, in particular from the point of view of the organoleptic properties and their percentage of alcohol. It makes it possible in particular to preserve or enhance the usual organoleptic properties of a beverage while reducing its percentage of alcohol. In other cases, it makes it possible to produce beverages having novel organoleptic characteristics or textures which cannot be obtained by conventional methods. There may be mentioned for example the production of wines with a high glycerol content which are called “ropy” wines.

The method according to the invention is thus applicable to any type of fermentation using yeasts or other microorganisms of the same species as yeasts where the reduction in the production of ethanol and/or the increase in the production of glycerol are desirable.

According to a variant embodiment, the method is applied to yeast fermentations carried out in a closed vessel (for example bottle or cask) for the production of beverages with secondary fermentation.

They involve for example fermentation by yeasts mainly of the genus Saccharomyces, carried out for the production of sparkling wines, beers or ciders.

In this case, under reducing conditions as defined according to the invention, it is possible to improve the kinetics of the secondary fermentation by accelerating the increase in pressure in the bottle.

It is thus possible to prepare beverages of the champagne, sparkling wine, semi-sparkling wine, beer or cider type by a method which is easier and more rapid.

According to other embodiments of the invention, the method is applied to microorganisms for the preparation of ferments, food yeasts or yeast extracts, used in the food sector but also in the medical or veterinary sector.

By way of examples, there may be mentioned the culture of species of the genera Saccharomyces and Candida for the production of ferments or leavens for breadmaking, the culture of species of the genera Saccharomyces, Kluyveromyces and Candida for the production of food yeasts, in particular for pharmaceutical or veterinary use, and yeast extracts; the culture of bacteria of the genera Lactococcus, Leuconostoc, Lactobacillus and Streptococcus thermophilus for the production of lactic acid bacteria used in the dairy industry.

When reducing conditions as defined in accordance with the invention are used, more storage sugars, in particular trehalose and glycogen, are for example produced.

The content of storage sugars is an important factor and even a critical parameter in the production of active dry yeasts which are obtained by a method of dehydrating the said yeasts.

A good viability of the microorganisms is observed in parallel under the reducing conditions of the invention.

The method described therefore makes it possible to improve the production of such dehydrated yeasts.

It is thus applicable in the dietetic sector where dry food yeasts are useful as natural food ingredients, rich in proteins, group B vitamins and minerals.

It is also applicable in pharmacy and parapharmacy where the enriched dry yeasts can be incorporated into nutritional supplements, in particular through the high bioavailability of the trace elements which they contain, and where the yeast extracts are useful in pharmaceutical fermentation but also in microbiology in particular for the preparation of growth media.

The method of the present invention is also applicable to the food sector where the autolysed yeasts are ingredients which are particularly well suited to flavouring, in particular for the preparation of flavoured snacks or savoury biscuits and where the yeast extracts are traditional ingredients in broths, vegetable soups and the like, but also ingredients rich in growth factors, in peptides and in amino acids which are useful for the methods of culturing microorganisms.

The method according to the invention is applicable to any industry using live leavens such as oenology, brewery, cidermaking, breadmaking but also pharmacy, parapharmacy, veterinary medicine, in particular with the rapid development of probiotic agents.

The invention thus relates to any product obtained by the method described above.

The invention is illustrated with the aid of the examples given below, with no limitation being implied, with reference to the drawings in which:

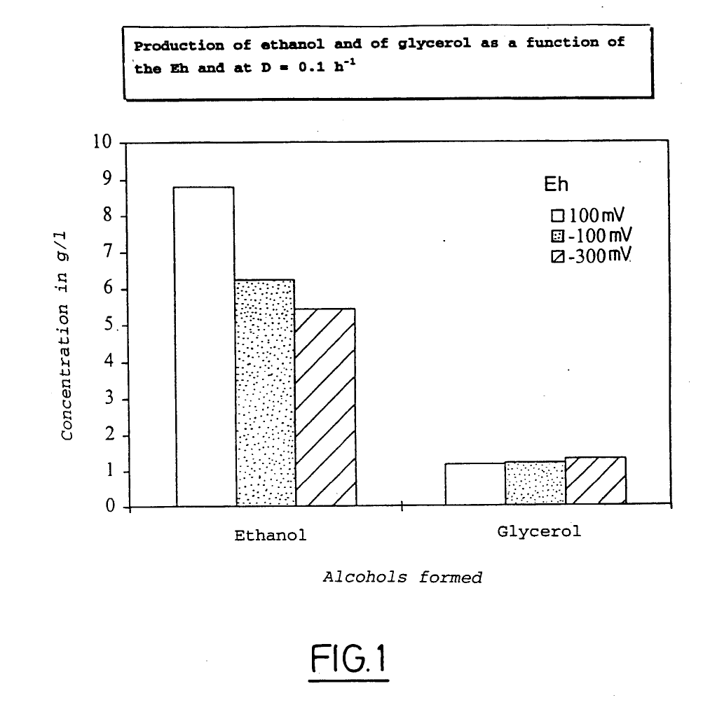

FIG. 1 is a diagram showing the production of ethanol and of glycerol obtained for different redox potentials (Eh) in the case of a continuous fermentation on minimum medium, with reference to Example 1;

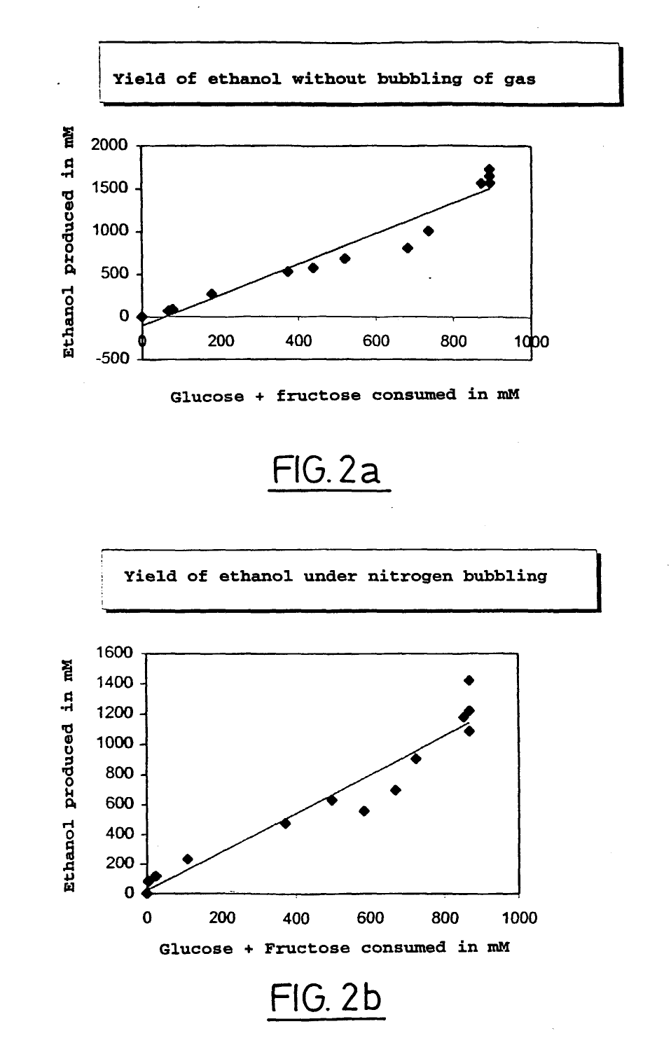

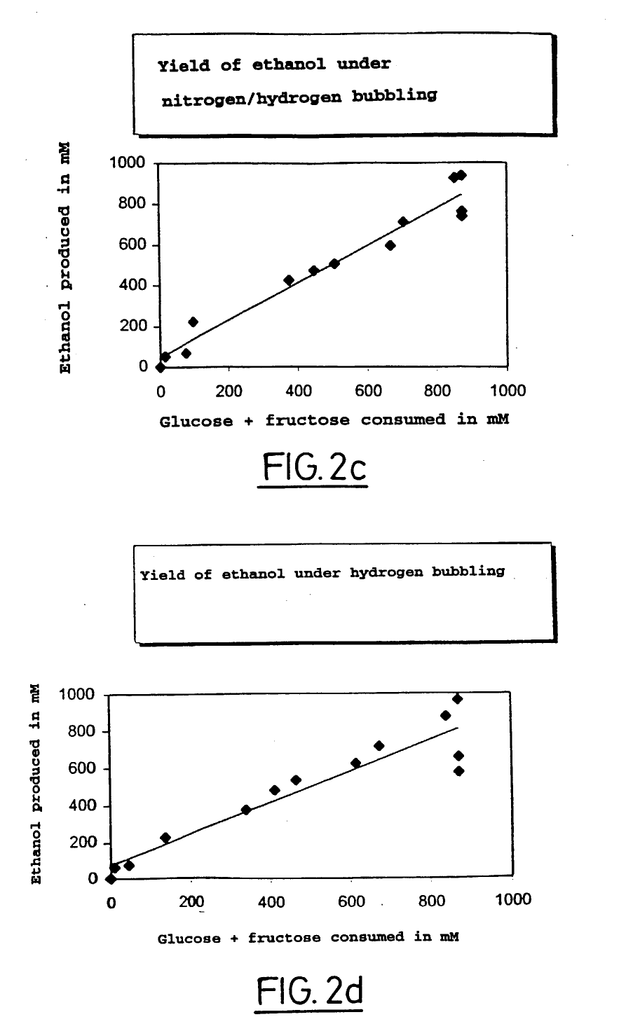

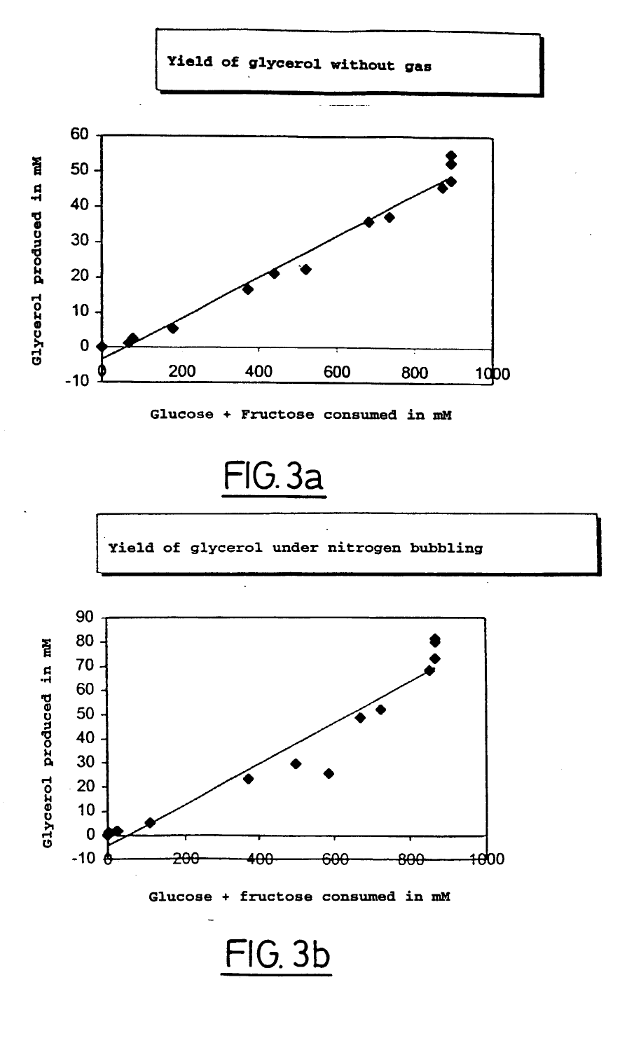

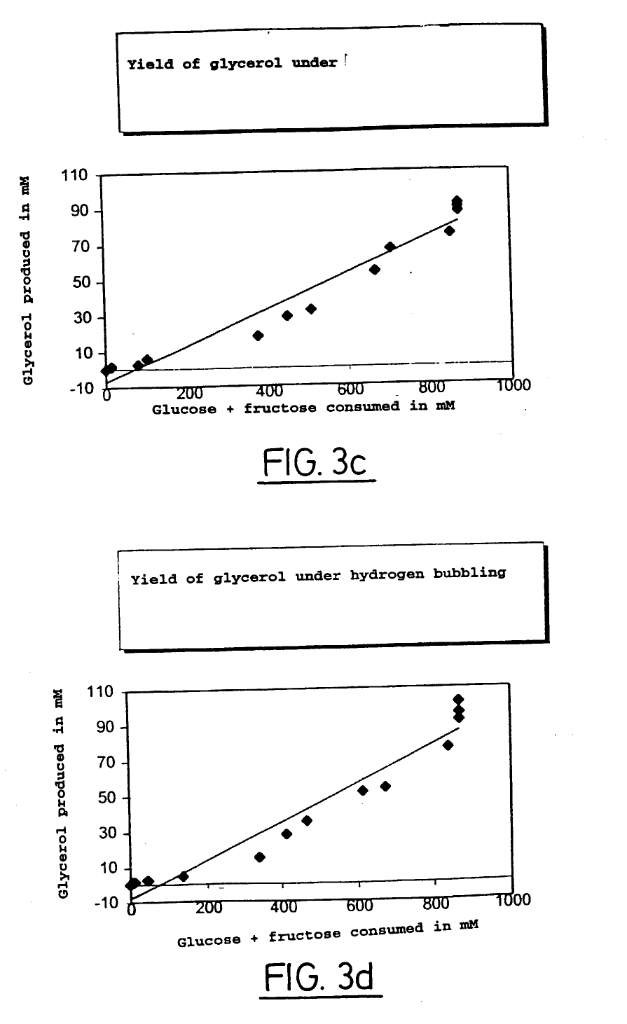

FIGS. 2a to 2d and 3a to 3d are curves showing respectively the yields of ethanol and of glycerol observed in the absence of gas and under three different bubblings (generating four different redox potentials) during an alcoholic fermentation, with reference to Example 2;

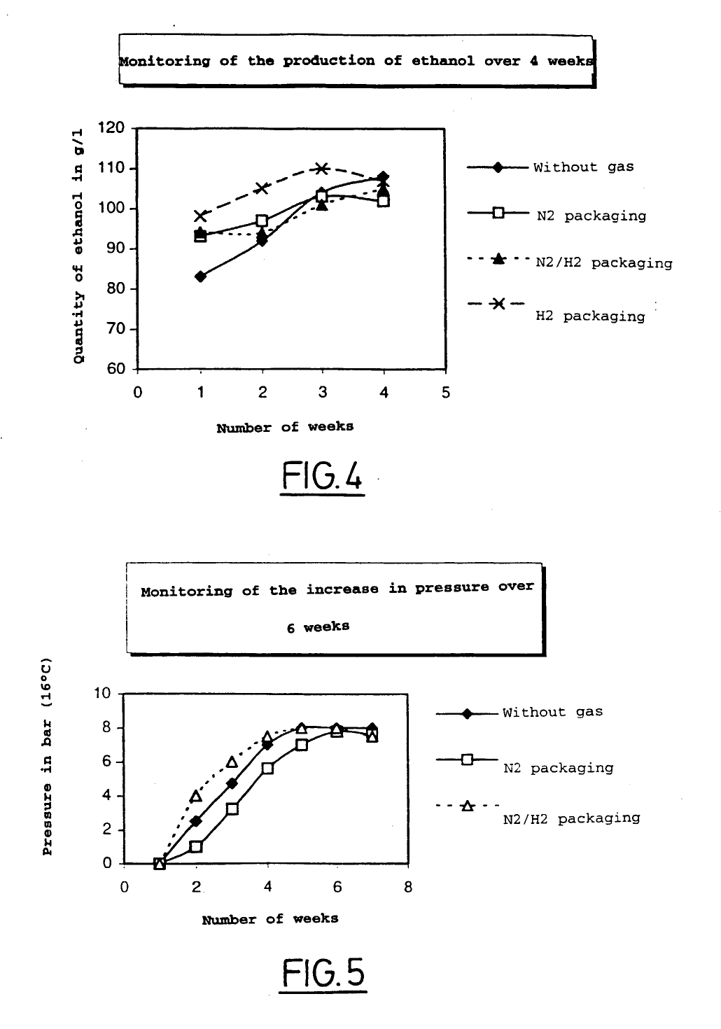

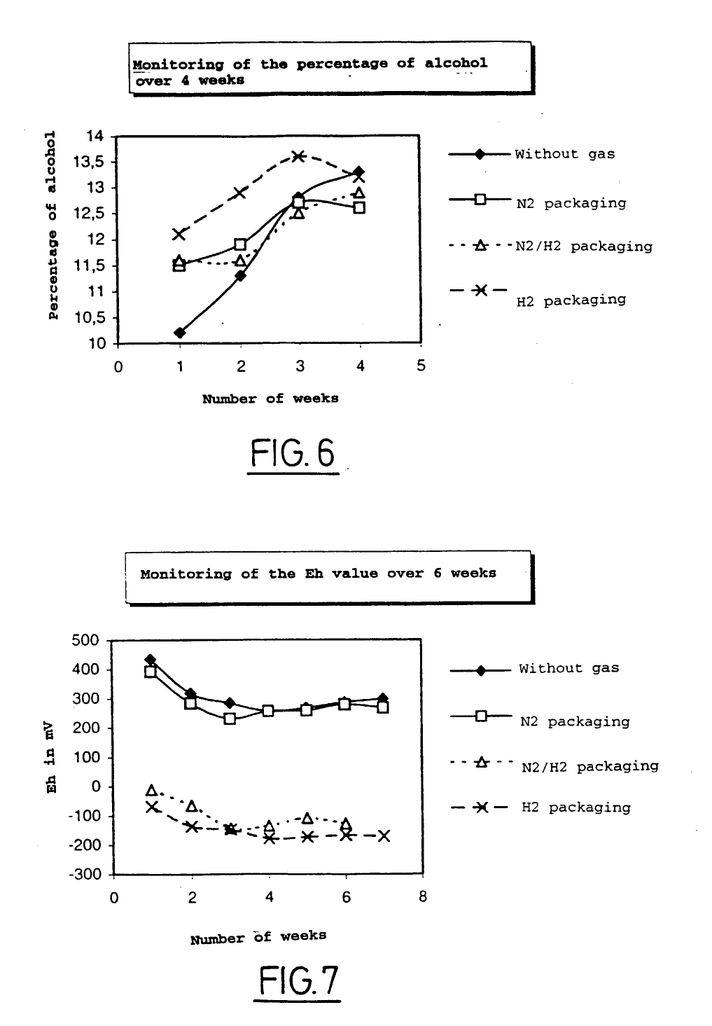

FIGS. 4, 5 and 6 represent the curves showing respectively the variation in the production of ethanol, in the pressure in the bottle and in the percentage of alcohol (decisive parameters in a secondary fermentation) as a function of time, in the case of a fermentation in bottles with secondary fermentation, with reference to Example 3;

FIGS. 7, 8 and 9 represent curves reporting respectively the redox potential (Eh), the monitoring of the quantity of residual sugar and the monitoring of the production of glycerol over time (ancillary parameters of a secondary fermentation) in the case of a fermentation in bottles with secondary fermentation, with reference to Example 3;

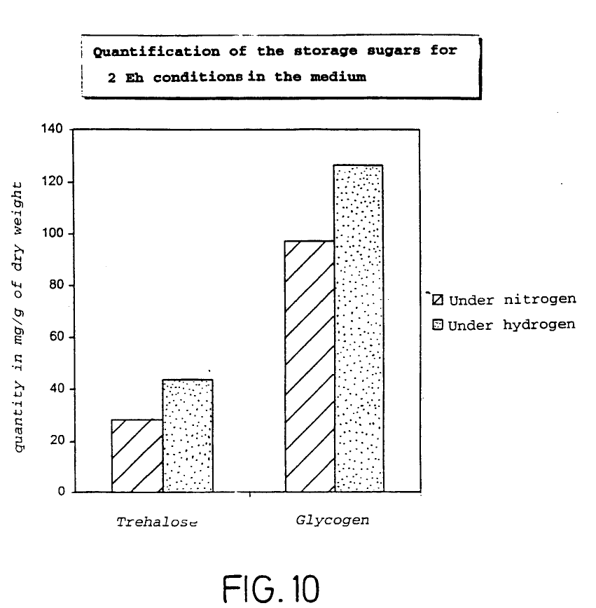

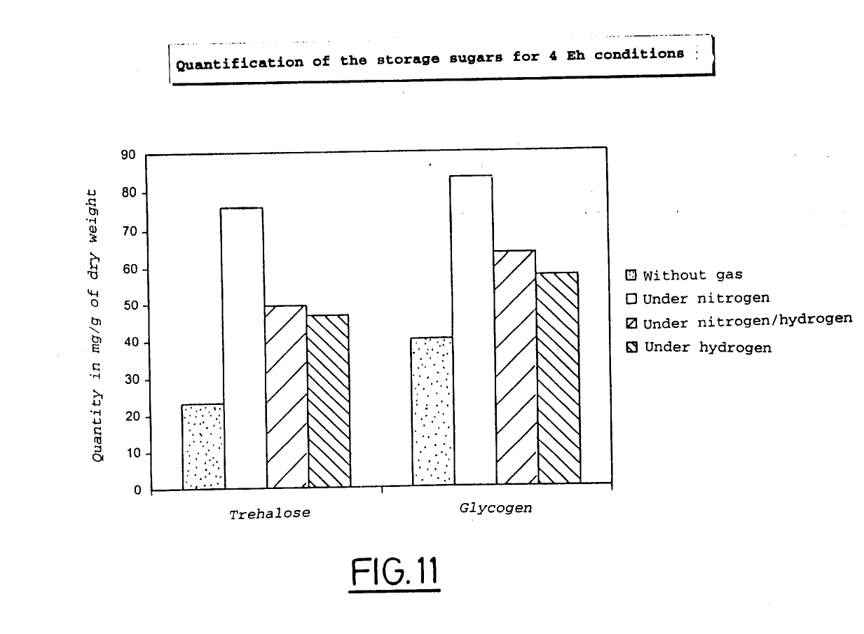

FIGS. 10 and 11 are diagrams illustrating the quantitative analysis of the storage sugars, glycogen and trehalose, as a function of the redox potential (Eh) respectively on minimum medium and on grape juice medium, with reference to Example 4;

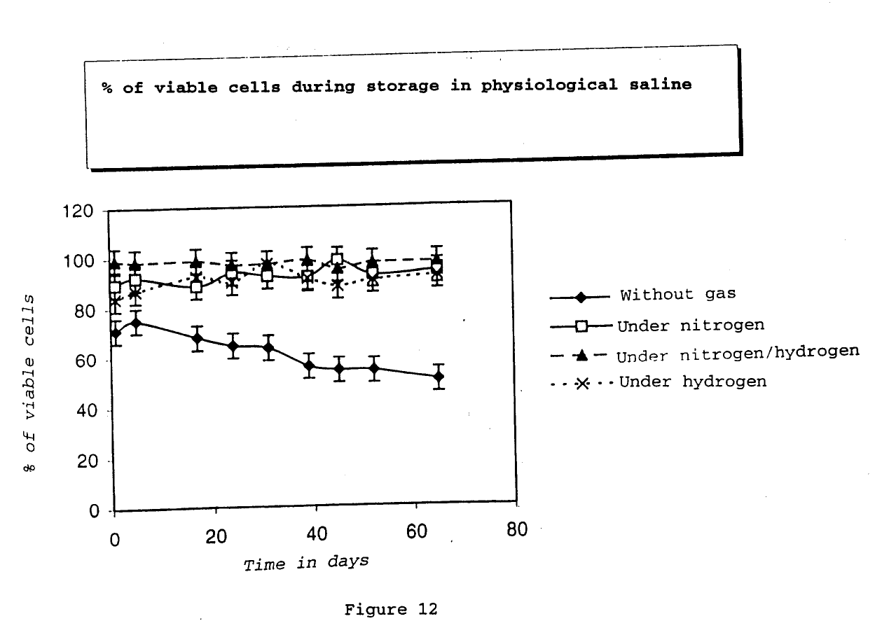

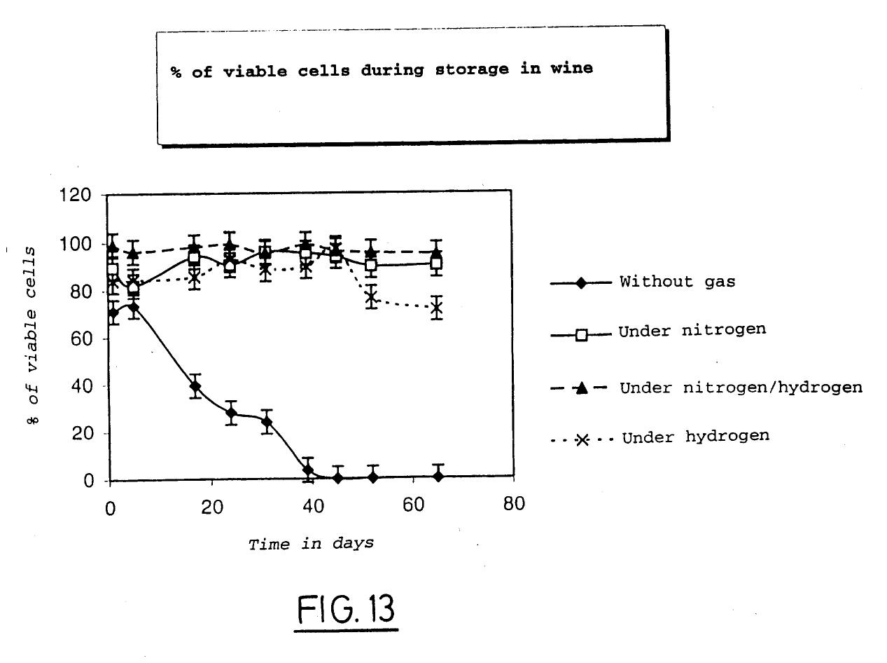

FIGS. 12 and 13 are curves showing the viability of the cells during their storage respectively in physiological saline and in wine, with reference to Example 5.

### EXAMPLES

### I—Effect Of The Reducing Conditions According To The Invention On The Value Of The Oxidation-Reduction Potential Of A Fermentation Medium

For this study, three types of atmospheres were used, namely nitrogen alone, nitrogen/hydrogen mixture in an amount of 4% of hydrogen and hydrogen alone.

The effect of a bubbling of these different gases on the value of the redox potential (Eh) of an inorganic medium having the following composition suitable for the growth of Saccharomyces cerevisiae was observed:

Composition of the inorganic medium:

Quantities for 1 litre of distilled water

| | | |
|---|---|---|
| (NH₄)₂SO₄ | 5 | g |
| KH₂PO₄ | 3 | g |
| MgSO₄.7H₂O | 0.5 | g |
| EDTA | 15 | mg |
| ZnSO₄.7H₂O | 4.5 | mg |
| CoCl₂.6H₂O | 0.3 | mg |
| CaCl₂.2H₂O | 4.5 | mg |
| FeSO₄.7H₂O | 3 | mg |
| NaMoO₄.2H₂O | 0.4 | mg |
| H₃BO₃ | 1 | mg |
| KCl | 0.1 | mg |
| Antifoaming silicone | 0.025 | ml |
| Ergosterol | 10 | mg |
| Tween 80 | 420 | mg |
| Ethanol 1 mM | 46 | mg |
| Glucose | 23 | g |
| Biotin | 0.05 | mg |
| Calcium pantothenate | 1 | mg |
| Nicotinic acid | 1 | mg |
| Inositol | 25 | mg |
| Thiamine HC1 | 1 | mg |
| Para-aminobenzoic acid | 0.2 | mg |
| Pyridoxine HC1 | 1 | mg |

pH = 4.1

The effect of these gases on grape juice medium corresponding to an oenological application was also observed.

In the first stage, the Eh value was observed on the sterile medium (that is to say noninoculated medium) and, in a second stage, on inoculated medium.

The gas or the gas mixture is bubbled through the fermentation medium until the Eh value is obtained. These experiments are carried out at 25° C. and at the pH of the medium considered which remains constant.

Table 1 below reports the values of the redox potential (Eh) obtained on minimum medium.

TABLE 1

| | Nitrogen | Nitrogen/hydrogen | Hydrogen |
|---|---|---|---|
| Sterile | +250 mV | −200 mV | −300 mV |
| medium | (pH = 4.13) | (pH = 4.14) | (pH = 5) |
| Inoculated | +100 to +70 mV | −100 mV | −300 mV |
| medium (*) | (pH = 5) | (pH = 5) | (pH = 5) |

(*) Saccharomyces cerevisiae CBS 8066 strain

Table 2 indicates, for its part, the values of the redox potential (Eh) obtained on grape juice medium.

TABLE 2

| | Nitrogen | Nitrogen/hydrogen | Hydrogen |
|---|---|---|---|
| Sterile | +300 mV | −100 mV | −200 mV |
| grape juice | (pH = 3.01) | (pH = 3.04) | (pH = 3.02) |
| Inoculated | +100 to +300 mV | −100 mV | −200 mV |
| grape juice (**) | (pH = 3) | (pH = 3) | (pH = 3) |

(**) Saccharomyces cerevisiae RC 212 strain

By way of comparison, the potential of the sterile grape juice medium is 400 mV under conventional conditions (absence of gaseous stream according to the invention).

### II—Effect Of The Reducing Conditions According To The Invention On The Metabolic Flows Of The Fermentation

### Example 1

### Study On Minimum Medium

The Saccharomyces cerevisiae CBS 8066 strain was used which develops on an inorganic medium in limiting glucose and supplemented with vitamins and unsaturated fatty acids corresponding to the composition given in paragraph I. The yeasts are continuously cultured under anaerobic conditions at a constant temperature of 30° C. and a pH maintained at 5. The dilution rates D (ratio of feed rate to volume of liquid) are scanned from 0.05 h⁻¹ to 0.3 h⁻¹.

Three values of Eh are applied using the different gases, that is 100 mV, −100 mV and −300 mV.

The modification of the metabolic flows, during the fermentation, is studied by quantitative analysis of the distribution of the metabolic flows:

The results observed are reported in FIG. 1 which presents the quantity (in g/l) of ethanol and of glycerol for the three conditions of Eh cited above at D=0.1 h⁻¹. In reduced medium, the carbon flow is deviated towards the production of glycerol at the expense of ethanol.

The results obtained during the monitoring of the metabolite-substrate stoichiometry are given in Table 3.

In the light of the results presented in this table, it is observed that under reducing conditions (hydrogen gas and hydrogen/nitrogen mixture), the synthesis of glycerol is increased compared with oxidizing conditions (under nitrogen), this being at the expense of that of ethanol which is greatly reduced. Under these conditions, the glycerol/ethanol ratio is doubled when there is a transition from a fermentation under nitrogen to a fermentation under hydrogen.

### Example 2

### Study On Grape Juice Medium

An oenological strain of Saccharomyces cerevisiae RC 212 was used. The fermentation is carried out batchwise for 145 hours on grape juice medium containing about 165 g/l of fermentable sugars and the temperature is maintained at 25° C. This is an industrial medium in which the physicochemical properties and the concentrations of nutritive substances are different from the inorganic medium. This medium has the capacity to reproduce the phenomenon at a high concentration of sugars and with a substrate composed of a balanced glucose-fructose mixture. This experiment also makes it possible to control the phenomenon in batch culture.

Four redox potential (Eh) conditions are tested using:

a reactor without bubbling of gas (FIGS. 2a and 3a)

a reactor with nitrogen bubbling (FIGS. 2b and 3b)

a reactor with nitrogen/hydrogen bubbling (FIGS. 2c and 3c)

a reactor with hydrogen bubbling (FIGS. [sic] and 3d).

Over time, the biomass, the pH, the variation in Eh, the production of ethanol and of glycerol as well as the quantity of residual sugars were monitored.

The results presented correspond to a mean of 3 repeats.

They are illustrated in FIGS. 2a to 2d for ethanol and in FIGS. 3a to 3d for glycerol.

The concentrations of sugars in the musts being variable (150 to 350 g/l of juice depending on the vine cultivars), the results obtained should be interpreted taking into account the ethanol formed/fermentable sugars (in mol/mol) and glycerol formed/fermentable sugars (in mol/mol) ratios but also the glycerol/ethanol ratio.

This analysis of the metabolite-sugars stoichiometry confirms, as in the study on minimum medium in a chemostat, the fact that the pathway for glycerol is favoured at the expense of that for ethanol when the yeast is under reducing conditions.

These results show that in the context of a winemaking application, it is possible to produce a fermented beverage with a low percentage of alcohol, while maintaining (or while increasing) the production of glycerol which plays a role in the body and fullness of wines.

Table 3 below gives the results obtained during the monitoring of the metabolite-substrate stoichiometry in the case:

(1) of a fermentation in a chemostat at D=0.1 h⁻¹ on minimum medium (23 g/l of glucose): Example 1,

(2) of a batch fermentation on grape juice at 100 hours (165 g/l of fermentable sugars): Example 2.

The reported yields correspond to the direction coefficients of the regression lines in FIGS. 2a-2d and 3a-3d:

FIG. 2a: y=1.79x−105.00; yield=94%

FIG. 2b: y=1.29x−23.47; yield=93%

FIG. 2c: y=0.91x−45.92; yield=96%

FIG. 2d: y=0.85x−72.42; yield=88%

FIG. 3a: y=0.06x−3.51; yield=98%

FIG. 3b: y=0.10x−6.92; yield=95%

FIG. 3c: y=0.09x−4.46; yield=93%

FIG. 3d: y=0.11x−8.08; yield=94%

By way of comparison, this table indicates the values reported by Michnick et al., 1997 (Yeast, vol. 13, 783-793) and Oura, 1977 (Process Biochemistry, vol. 4, 19-35) in the prior art, the second reference corresponding to a summary by 5 different authors.

TABLE 3

| | Conditions | | | | | | | | | | | |
|---|---|---|---|---|---|---|---|---|---|---|---|---|
| | Ethanol/sugars | | | | | | Glycerol/sugars | | | | | |
| | in mol/mol (A) | | | | | | in mol/mol (B) | | | | | |
| | Control | N₂ | N₂/H₂ | H₂ | Ratios | | Control | N₂ | N₂/H₂ | H₂ | Ratios | |
| Data | (1) | (2) | | (3) | (3)/(1) | (3) (2) | (1) | (2) | (3) | | (3)/(1) | (3) (2) |
| Chemostat | | 1.50 | 1.06 | 0.93 | | 0.62 | | 0.1 | 0.1 | 0.11 | | 1.1 |
| fermentation | | | | | | | | | | | | |
| at D = 0.1 h¹ | | | | | | | | | | | | |
| Batch | 1.79 | 1.29 | 0.91 | 0.85 | 0.47 | 0.66 | 0.06 | 0.09 | 0.10 | 0.11 | 1.83 | 1.22 |
| fermentation | | | | | | | | | | | | |
| on grape juice | | | | | | | | | | | | |
| (at 100 hours) | | | | | | | | | | | | |
| Bibliographic | | | | | | | | | | | | |
| data | | | | | | | | | | | | |
| Michnick | 1.74 | | | | | | 0.07 | | | | | |
| (1997) | | | | | | | | | | | | |

| | | |
|---|---|---|
| Oura (1977) | 1.71-1.89 | 0.10-0.06 |

| | Conditions | | | | | |
|---|---|---|---|---|---|---|
| | Ratio B/A: | | | | | |
| | glycerol/ethanol | | | | | |
| | Control | N₂ | N₂/H₂ | H₂ | Ratios | |
| Data | (1) | (2) | (3) | | (3)/(1) | (3) (2) |
| Chemostat | | 0.06 | 0.09 | 0.11 | | 2 |
| fermentation | | | | | | |
| at D = 0.1 h¹ | | | | | | |
| Batch | 0.03 | 0.07 | 0.11 | 0.13 | 4.3 | 1.86 |
| fermentation | | | | | | |
| on grape juice | | | | | | |
| (at 100 hours) | | | | | | |
| Bibliographic | | | | | | |
| data | | | | | | |
| Michnick | 0.04 | | | | | |
| (1997) | | | | | | |

| | |
|---|---|
| Oura (1977) | 0.03-0.06 |

Control: without modification of the redox
(a) and (b): control for conventional conditions (without modification of the redox) and in batch culture

Control: without modification of the redox

(a) and (b): control for conventional conditions (without modification of the redox) and in batch culture

### Example 3

### Study On Champagne Medium

An oenological starter yeast strain of the genus Saccharomyces is used. The medium consists of a basic wine supplemented with a liquor enriched with sugars.

Four Eh conditions are tested using:

bottling without bubbling of gas,

bottling with nitrogen bubbling,

bottling with nitrogen/hydrogen bubbling,

bottling with hydrogen bubbling.

Over time (6 weeks), the Eh variation, the production of ethanol and glycerol, the viability of the yeast cells, the variation in pressure as well as the quantity of residual sugars (glucose+fructose) were monitored.

Monitoring of the decisive parameters for evaluating the secondary fermentation:

This in fact involves the measurement of the ethanol production, of the variation in pressure and the calculation of the percentage of alcohol obtained.

In the light of the curves presented in FIGS. 4, 5 and 6, it is observed that after 3 weeks, 12.5% is obtained in the bottles regardless of the Eh conditions tested. It is recalled that according to the legislation, this value is required for any manufacture of champagne.

Monitoring of the ancillary parameters:

This involves the measurement of the Eh and of the quantities of glycerol and residual sugars.

With reference to FIG. 7, the measurement of the Eh over 6 weeks makes it possible to conclude that the value is maintained in the bottles over time.

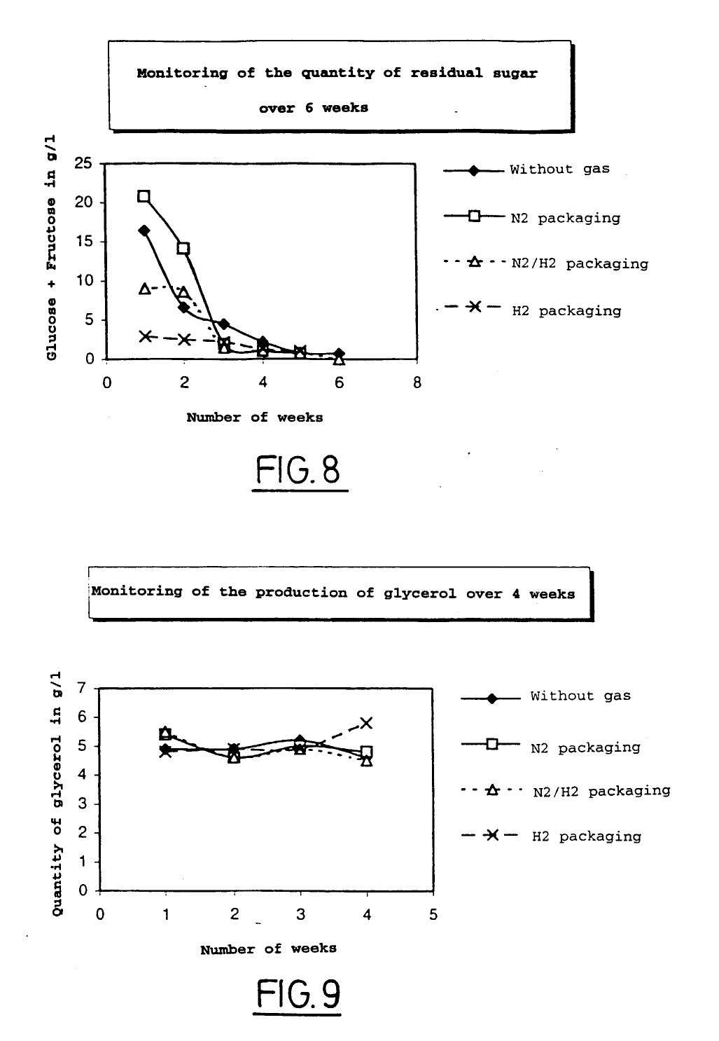

As regards the quantity of residual sugars, in the light of the curves of FIG. 8, depending on the Eh conditions tested, the consumption of sugar by the yeast does not occur in the same manner. The strain placed under reducing conditions consumes the fermentable sugars much more rapidly than under an oxidizing environment.

Finally, with reference to FIG. 9, it can be seen that the production of glycerol is not substantially modified by the different conditions applied.

This study shows the change in the physicochemisty of the product during the fermentation in bottles because of the modification of the Eh using different reducing conditions.

### III—Effect Of The Reducing Conditions According To The Invention On The Content Of Storage Sugars

### Example 4

### Study On Minimum Medium

Saccharomyces cerevisisae CBS 8066 was grown batchwise on a glucose-limiting inorganic medium whose composition corresponds to that given in I, at 30° C. and with stirring at 300 rpm (revolution per minute).

Two Eh conditions are tested:

a reactor under nitrogen bubbling,

a reactor under hydrogen bubbling.

The growth is carried out for 14 hours, following which a cell sample is collected in order to carry out the assay of the trehalose and of the glycogen according to the protocol established by Parrou and Francois, 1997 (Analytical Biochemistry, vol. 248, 186-188).

The results presented correspond to a mean of 2 assays.

The quantitative analysis of the accumulation of the storage sugars inside the yeast cell reported in FIG. 10 shows that in the presence of reducing conditions according to the invention, the strain synthesizes storage oligosaccharides in a larger quantity than under oxidizing conditions in response to this modification of the physicochemical environment.

### Example 5

### Study On Grape Juice Medium

An oenological strain of Saccharomyces cerevisisae RC 212 was used. The fermentation is carried out batchwise for 145 hours on grape juice medium containing about 165 g/l of fermentable sugars and the temperature is maintained at 25° C.

Four Eh conditions are tested using:

a reactor without bubbling of gas

a reactor with nitrogen bubbling

a reactor with a nitrogen/hydrogen bubbling (conditions according to the invention)

a reactor with a hydrogen bubbling (conditions according to the invention).

The cells were collected for the assay of the trehalose and of the glycogen after 128 hours of alcoholic fermentation, when practically all the fermentable sugars have been consumed (about 99.9%).

The viability of the cells to methylene blue was measured over time. The samples collected are then stored at 4° C. without any special precaution except that a portion of the cells is taken up in physiological saline and the other portion in the wine medium from which they were collected. The viability was monitored over time.

Assay of the trehalose and of the glycogen:

The results presented correspond to a mean of 2 repeats.

From the quantitative analysis illustrated in FIG. 11, it can be noted that the yeast accumulates more storage sugars when it is under reducing conditions in accordance with the invention rather than under oxidizing conditions.

It is observed that this accumulation is optimum on the medium placed under nitrogen, a nonreducing gas but which leads to a reduction in the redox potential of the medium compared with the value which it would have had in the absence of gas (nitrogen).

Monitoring of the viability over time:

In the light of the curves given in FIGS. 12 and 13, it is very advantageous to note that the viability is optimum for the cells resulting from the reactor placed under nitrogen/hydrogen. In this case, the most optimum for allowing better storage of the yeast cells over time.

1. Method of culturing microorganisms which makes it possible to reduce the oxidation-reduction potential of the culture medium, characterized in that the said culture is carried out under reducing conditions obtained by a reducing gas compared with air comprising hydrogen.

2. Method of culturing microorganisms which makes it possible to modify the metabolic flows during the said culture, characterized in that the said culture of microorganisms is carried out under reducing conditions obtained by a reducing gas compared with air comprising hydrogen.

3. Method according to either of claims 1 and 2, characterized in that it consists of a fermentation using yeasts.

4. Method according to claim 3, characterized in that it is intended for the production of non-aerated beverages with a reduced percentage of alcohol by fermentation using yeasts in open vessels, in particular vats, in particular for the production of wine-based beverages and of distilled beverages.

5. Method according to claim 4, characterized in that the quantity of glycerol produced is increased and the quantity of ethanol produced is reduced.

6. Method according to claim 5, characterized in that the glycerol/ethanol ratio is multiplied by a factor of 1.5 to 5 under reducing conditions, preferably 2 to 4.

7. Method according to claim 3, characterized in that it is intended for the production of aerated alcoholic beverages by fermentation using yeasts in closed vessels, in particular in bottles or casks, in particular for the production of semi-sparkling wines, sparkling wines, champagnes, ciders and beers.

8. Method according to claim 2 or 3, characterized in that it is intended to increase the quantity of storage sugars, in particular trehalose and glycogen, produced during the said culture.

9. Method according to claim 8, characterized in that it is intended for the production of ferments, in particular leavens for breadmaking, food yeasts and yeast extracts.

10. Method according to any one of the preceding claims, characterized in that the culture of the microorganisms is carried out under a hydrogen gas.

11. Method according to any one of claims 1 to 9, characterized in that the culture of the microorganisms is carried out under a gas consisting of a mixture of hydrogen and nitrogen.

12. Method according to any one of claims 1 to 9, characterized in that the culture of the microorganisms is carried out under a reducing gas compared with air, containing hydrogen and nitrogen and a supplementary gas acceptable from the point of view of the said culture.

13. Method according to claim 12, characterized in that the gas comprises at least 0.5% by volume of hydrogen, preferably between 3 and 50% by volume of hydrogen.

14. Method according to either of claims 12 and 13, characterized in that the reducing gas has a hydrogen content of less than 5% by volume.

15. Method according to any one of claims 12 to 14, characterized in that the supplementary gas is chosen from inert gases, in particular argon, helium, and from oxygen, carbon dioxide, nitrous oxide and mixtures thereof in any proportions, preferably from carbon dioxide and oxygen as well as mixtures thereof.

16. Method according to any one of claims 12 to 15, characterized in that the culture of the microorganisms is carried out under a gas containing hydrogen and carbon dioxide.

17. Method according to claim 3, characterized in that the yeasts are of the genus Saccharomyces.

18. Use of a reducing gas compared with air, comprising hydrogen, for reducing the oxidation-reduction potential of a medium for culturing microorganisms.

19. Use according to claim 18, for modifying the metabolic flows during the culture of microorganisms.

20. Use according to either of claims 18 and 19, in a fermentation process using yeasts.

21. Use according to any one of claims 18 to 20, for the production of non-aerated beverages with a reduced percentage of alcohol by fermentation using yeasts in open vessels, in particular in vats, in particular for the production of wine-based -beverages and of distilled beverages.

22. Use according to any one of claims 18 to 20, to increase the quantity of glycerol produced and to reduce the quantity of ethanol produced.

23. Use according to claim 22, to obtain a glycerol/ethanol ratio multiplied by a factor of 1.5 to 5 under reducing conditions, preferably 2 to 4.

24. Use according to any one of claims 18 to 20, for the production of aerated alcoholic beverages by fermentation using yeasts in closed vessels, in particular in bottles or casks, in particular for the production of semi-sparkling wines, sparkling wines, champagnes, ciders and beers.

25. Use according to any one of claims 18 to 20, to increase the quantity of storage sugars, in particular trehalose and glycogen, produced during the said culture.

26. Use according to any one of claims 18 to 20 and 25, for the production of ferments, in particular leavens for breadmaking, food yeasts and yeast extracts.

27. Use according to any one of claims 18 to 26, characterized in that the reducing gas consists of hydrogen.

28. Use according to any one of claims 18 to 26, characterized in that the reducing gas consists of a mixture of hydrogen and nitrogen.

29. Use according to any one of claims 18 to 26, characterized in that the reducing gas compared with air contains hydrogen and nitrogen and a supplementary gas acceptable from the point of view of the culture of the microorganisms.

30. Use according to claim 29, characterized in that the reducing gas contains at least 0.5% by volume of hydrogen, preferably between 3 and 50% by volume of hydrogen.

31. Use according to either of claims 29 and 30, characterized in that the reducing gas has a hydrogen content of less than 5% by volume.

32. Use according to any one of claims 29 to 31, characterized in that the supplementary gas is chosen from inert gases, in particular argon, helium, and from oxygen, carbon dioxide, nitrous oxide and mixtures thereof in any proportions, preferably from carbon dioxide and oxygen as well as mixtures thereof.

33. Use according to any one of claims 29 to 32, characterized in that the reducing gas contains hydrogen and carbon dioxide.

34. Ferments, in particular leavens for breadmaking, food yeasts and yeast extracts, obtained according to a method in accordance with any one of claims 1, 2, 3, and 8 to 17.

35. Non-aerated beverages with a reduced percentage of alcohol, in particular wine-based beverages and distilled beverages, aerated alcoholic beverages, in particular semi-sparkling wines, sparkling wines, champagnes, beers and ciders, obtained according to a method in accordance with any one of claims 1 to 8 and 10 to 17.
